(19) United States
(12) Patent Application Publication
(10) Pub. No.: US 2021/0011018 A1
(43) Pub. Date: Jan. 14, 2021

(54) ADVANCED BIOPHYSICAL AND BIOCHEMICAL CELLULAR MONITORING AND QUANTIFICATION USING LASER FORCE CYTOLOGY

(71) Applicant: Lumacyte, LLC, Charlottesville, VA (US)

(72) Inventors: Sean Hart, Keswick, VA (US); Colin Hebert, Charlottesville, VA (US); Margaret McCoy, Charlottesville, VA (US)

(21) Appl. No.: 16/982,935

(22) PCT Filed: Mar. 20, 2019
(86) PCT No.: PCT/US2019/023130

§ 371 Date: Sep. 21, 2020

Related U.S. Application Data
(60) Provisional application No. 62/645,652, filed on Mar. 20, 2018.

(51) Int. Cl. G01N 33/569; G01N 15/14

(52) U.S. Cl. CPC G01N 33/56983; G01N 2015/1006; G01N 15/1434

(57) ABSTRACT

The present invention is directed to intelligent algorithms, methodologies and computer-implemented methodologies for biophysical and biochemical cellular monitoring and quantification enabling enhanced performance and objective analysis of advanced infectivity assays including neutralization assays and adventitious agent testing using fluidic and optical force-based measurements.

### FIELD OF THE INVENTION

Embodiments of the present disclosure relate generally to measuring cellular responses to differential stimuli utilizing optical and/or fluidic forces, as well as intelligent algorithms (IA) resulting in methodologies for biophysical and biochemical cellular monitoring and quantification; in certain embodiments, the methodologies herein are computer-implemented. The embodiments described herein include the enablement of enhanced performance and objective analysis of advanced infectivity assays including neutralization assays and adventitious agent testing (AAT). The methods as described use optical force-based measurements, such as laser force cytology (LFC). Specifically, the current disclosure describes an automated algorithm and infection metric calculations for the automated scanning and analysis of multi-well plates for neutralization and other functional assays. Additionally, the use of suspension or matrix-embedded cells are enabled in order to expand the infection models that can be utilized for such assays as well as the ability to monitor, assess, and quantify adventitious agent (AA) samples and cultures.

### BACKGROUND OF THE INVENTION

Currently, the serum virus neutralization assay is the gold standard for analysis of the ability of in vivo-derived immunity to inhibit viral infection and/or replication. Neutralization assays are used to determine the efficacy of serum-derived antibodies to reduce or block viral infection and/or subsequent replication in cells in culture. Basically, human or animal cells are treated in vitro with combinations of infectious viral agents and in vivo-derived serum antibodies in order to examine whether the serum-derived antibodies are specific for and effective against the infection and/or replication of the viral agent within the cells in vitro. Additional analysis is required for these types of analytical experiments. The plaque assay and plaque reduction neutralization test (PRNT) both measure the number of infectious viral particles per unit volume of sample, the latter also measuring the reduction in infectious units as a result of a neutralizing serum or other agent. The assay involves placing a virus containing solution on growing adherent cells in a plate, applying an overlay (typically agarose) to prevent the free spread of virus and then waiting between 3 and 15 days for regions of dead or cleared cells (plaques) to develop as a result of a single infectious virus particle. Similarly, the tissue culture infectious dose 50 (TCID50) is a measure of the concentration of infectious virus in a specific volume by performing the endpoint dilution assay. The TCID50 is defined as the dilution of virus required to infect 50% of a given batch of inoculated wells of cells in culture. Though these methods have been used for decades, there are inherent challenges to performing them with reliability and reproducibility of results between experiments and operators. There are also limitations of the assays with respect to analyzing cells in suspension, requirements for a high number of samples (for dilution calculations), time-consuming and subjective techniques for analysis and undesirable consequences such as cell death and/or alteration of infection parameters resulting from cell manipulations. One reason for the large number of required dilutions is the limited dynamic range of current methodologies and the high variability of current methodologies.

The prior art describes a method and apparatus for using optical density and various constraints to determine a neutralization titer such as analyzing and plotting the maximum optical density of each sample (U.S. Patent Publication No. 2013/0084560, which is incorporated herein by reference). U.S. Patent Publication No. 2013/0084560 however only uses optical density and does not utilize microfluidic and/or optical forces, and neither does it incorporate the use of additional intelligence by utilizing an automatic real-time grid search algorithm to calculate which samples need to be read/analyzed in order to determine the results of the experiment. Another semi-automated system is described in U.S. Pat. No. 4,329,424 however this methodology utilizes a light source, not optical forces, and is not fully automated.

Additionally, whereas U.S. Pat. No. 8,778,347 describes the use of inactivated fluorescently-labeled virus monitored by flow cytometry in order to reduce the safety precautions required for experimental manipulation, and European Patent No. 1140974 describes the use of a pseudovirion reporter gene, both references are limited in that large numbers of samples must be analyzed due to cumbersome tagging or modification of sample cells or infectious agents used in the assays. As modification of cells and infectious agents has been shown to activate, differentiate, or alter infectivity and/or function, what is needed is label-free analysis as an ideal alternative to the traditional methods which require such modifications for analysis.

Furthermore, whereas WO1989006705 describes the use of a plaque transfer assay for detecting retrovirus and measuring neutralizing antibodies, the teachings therein limit the experimenter to the use of a monolayer cell types only. In reality, as is well known to those skilled in the art, not all viruses infect cells that form a monolayer. What is needed are methods and devices that enable the use of suspension or matrix-embedded cells for infection study and analysis thereby allowing a larger variety of cell types to be used in experimentation for viral infection.

The prior art such as U.S. Pat. No. 6,778,263 describes the use of calibration objects (e.g., beads or cells), however, such teachings are limited in that they describe the use of calibration objects in the context of a time-delay-integration (TDI) detector only. Functionality of the TDI detector relies on shifting the lines of photon-induced charge in the solid-state detector (such as a charge-couple device array) in synchronization of the flow of the specimen, and the calibration objects are used to enhance the performance of this system. Furthermore, not only are the calibration beads of the prior art limited to calibrating flow and aligning TDI detectors, they are not used to calibrate analytical information for data correction, normalization, quantitation, or calculations of physical or chemical information such as refractive index (ratio of refractive indices of bead/artificial cells, for example). What is lacking is the teaching or use of calibration objects that describe measurements such as optical force, optical torque, optical dynamics, effective refractive index, size, shape, or related measurements wherein said objects are polymer, glass, biologic, lipid, vesicles, or cell (live or fixed) based. Furthermore, what is also lacking is a teaching of calibration objects having properties related to the particles of interest, yet not interfering with data collection on samples of interest.

What is needed are improved methods and devices for efficiently characterizing biological components and systems with respect to numerous identifying aspects such as biophysical and biochemical profiles. In certain embodiments, such methods and devices should comprise intelligent algorithms and methodologies applicable to samples such as those derived from viral-based vaccination or drug discovery trials enabling whole or depleted cell isolates to be examined for infectivity parameter deviations between cell types, between groups of subjects or even between trials. Other sample treatments could include, the assessment of serum antibodies, antiviral compounds, antibacterial compounds, toxins, toxic industrial materials or chemicals (TIMs/TICs), parasites, and gene or cell therapy products such as CAR T-cells and oncolytic vaccines. What is also needed are neutralization assays for bacteria utilizing cells designed to be sensitive to bacteria (low response threshold) including cell lines or primary cells used to measure the infectivity of an infectious agent using multifaceted optical force-based measurements. Such methods and devices should ideally enable the determination of infectivity measurements useful for adventitious agent testing through the analysis of biomanufacturing liquids such as conditioned media or another samples of interest such as those obtained from bioreactors or other such vessels.

### SUMMARY OF THE INVENTION

Currently available procedural and analytical methodologies for the characterization of biological cells and systems such as infectivity assays (e.g., neutralization assays, TCID50 and clinical sample manipulation) require extensive dilutions, potentially detrimental tagging procedures and yield highly variable results making inter- and intra-experimental and trial comparisons challenging and downstream cellular applications limited. The current invention overcomes such limitations by providing novel methods related to biophysical and biochemical cellular monitoring and quantification including intelligent analytical algorithms for enhanced automated scanning of un-tagged cell samples using optical force-based technologies (such as laser force cytology (LFC)) that result in reduced requirements for sample dilutions, and ultimately sample specimens, as well as the time required for analysis and associated costs while enabling normalized and consistent evaluation of cells during analysis. Further, the present disclosure enables the use of suspension or matrix-embedded cells for analysis, expanding the dynamic range of infection models for neutralization or other functional assays as well as the ability to monitor, assess, and quantify adventitious agents from samples and cultures. Additionally, the inventive methods described herein may be computer-implemented thereby improving efficiency, reliability and reproducibility.

The basic premise of the background technology, laser force cytology (LFC), is that it utilizes the combination of microfluidics and light-induced pressure to take optical measurements including optical force or pressure, size, velocity, and other parameters on a per cell basis. While LFC is one preferred embodiment, other optical force-based technologies may be used according to the present invention. The application of LFC to the scanning and analysis of neutralization, TCID50, and other assays for determining viral titer and infectivity (both are synonymous with one another) and concentration determinations is performed by measuring changes in characteristics of cells that are indicative of the cytopathic effects of cells co-cultured with serum containing antibodies and/or a virus of interest as compared to cells treated with non-immunized serum alone (control or placebo). Additionally, cells co-cultured with a virus in the absence of serum can be used to determine the infection rate of cells derived from primary or cell culture sources. Hereinafter, any reference to neutralization assays will also be considered to include reference to TCID50 or plaque assay as the conventional application.

The current invention reduces the challenges associated with experimental subjectivity, time, and cost requirements while enhancing the objective ease of use with regards to reading and analyzing samples. This is enabled by using intelligent algorithms (IA) to scan and automatically and algorithmically calculate dilution and/or titer determinations and requirements, independent of human calculation and enabled by computer-implemented processes in certain embodiments. An intelligent algorithm is one that involves a complex set of instructions including fuzzy logic methods that encompass variable results such as infectivity and infection metrics (low, medium, or high infectivity ranges for example). The IA may also include artificial intelligence (AI) concepts including neural networks (NN) (back propagation or probabilistic NN) or machine learning to apply calibration data to the current samples to better predict the optimal grid search pattern for sampling. This novel methodologies disclosed herein ultimately reduce the number of dilutions required per experiment and thus save the experimenter resources, time, and the need for analysis of results by highly trained personnel, as well as eliminate the use of reporter genes, antibodies, or other staining/labeling mechanisms, as are currently required for quantification of neutralization assay titers.

The present invention optimizes the measuring of cellular responses to differential stimuli using optical and/or fluidic forces, and enables the delivery of consistent and reliable characterization of biological systems.

### BRIEF DESCRIPTION OF THE DRAWINGS

FIG. 1 is an example of the intelligent algorithm (IA) process for selecting sequential dilutions (100) and calculating TCID50/mL or percent neutralization on cell culture Well plates and defining the results as an Infection Metric/mL “IM” (120). Additionally, the IA (100) enables interpolation between dilutions and replicates using quantitative measurement of percent cytopathic effect (% CPE) of cells and analysis of the results (140).

FIG. 2 depicts a diagram detailing how an embodiment of the optical force-based technology, Radiance™, manipulates sample-containing culture plates utilizing (100) as described in this disclosure in FIG. 1. for application to neutralization (200) and TCID50 (220) assays.

FIG. 3 is a schematic demonstrating the use of calibration beads added to cell samples which may be used as an internal calibration standard.

FIG. 4. depicts the use of Radiance™ for bioreactor sampling and analysis for adventitious agent testing (AAT).

FIG. 5 illustrates a strategy AAT assessment and monitoring using Radiance™.

FIG. 6 is a summary table of virus CPE and replication in CHO cells.

FIG. 7 defines the potential for an LFC multiplexed assay using multiple cell types simultaneously for AAT.

FIG. 8 represents LFC analysis for AAT by sampling directly from a large process bioreactor.

FIG. 9 is a depiction of LFC analysis for AAT using mini-bioreactors running suspension cells spiked with CM.

FIG. 10 is a schematic illustrating LFC macrophage assay for AAT.

FIG. 11 provides a summary of discussing the development of an intelligent algorithm as used herein.

FIG. 12 provides a provides a flow chart demonstrating the intelligent algorithm as used herein (IM is Infection Metric, OLDR is Optimal Linear Dynamic Range).

FIG. 13 provides graphs demonstrating potential cases on which to apply intelligent algorithm: FIG. 13(A) Mid titer, FIG. 13(B) High titer, FIG. 13(C) Low titer, FIG. 13(D) Low titer (too much dilution), and FIG. 13(E) High titer (not enough dilution).

FIG. 14 provides a summary for calculating a titer and creating a calibration curve from a known viral system with a sample of unknown titer.

FIG. 15 provides a summary for calculating a titer and creating a calibration curve from an unknown (or not well understood) viral system with a sample of unknown titer.

FIG. 16 provides graphs showing infection metric vs. MOI for vero cells infected with vesicular stomatitis virus: FIG. 16. (A) MOI 0.125, FIG. 16(B) MOI 0.5, and FIG. 16(C) MOI 4.

FIG. 17 provides example data in four graphs demonstrating various measurements of adenovirus infection (Ad5) in adherent HEK 293 cells: FIG. 17(A) a scatter plot of size vs velocity, FIG. 17(B) a histogram showing velocity frequency, FIG. 17(C) a bar plot showing the multivariate infection metric for a range of MOI values, and FIG. 17(D) a scatter plot correlating the multivariate infection metric to the viral titer in PFU/mL.

FIG. 18 provides K-means cluster analysis of Radiance™ data.

FIG. 19 provides a schematic for calculating absolute titer/infectivity.

FIG. 20 provides graphs FIG. 20(A) titer (log scale), FIG. 20(B) titer (linear scale), and FIG. 20(C) infection metric.

FIG. 21 provides a graph demonstrating infectivity and absolute titer results.

FIG. 22 provides LFC identification of viruses using an ANN.

FIG. 23 provides a schematic summarizing steps for assessing cell responses as biomarkers for disease detection or vaccine efficacy for a placebo patient.

FIG. 24 provides a schematic summarizing steps for assessing cell responses as biomarkers for disease detection or vaccine efficacy for a patient subject.

### DETAILED DESCRIPTION OF THE INVENTION

The present invention is described with reference to particular embodiments having various features. It will be apparent to those skilled in the art that various modifications and variations can be made in the practice of the present invention without departing from the scope or spirit of the invention. One skilled in the art will recognize that these features may be used singularly or in any combination based on the requirements and specifications of a given application or design. One skilled in the art will recognize that the systems and devices of embodiments of the invention can be used with any of the methods of the invention and that any methods of the invention can be performed using any of the systems and devices of the invention. Embodiments comprising various features may also consist of or consist essentially of those various features. Other embodiments of the invention will be apparent to those skilled in the art from consideration of the specification and practice of the invention. The description of the invention provided is merely exemplary in nature and, thus, variations that do not depart from the essence of the invention are intended to be within the scope of the invention.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Unless otherwise defined, all technical and scientific terms used herein have the same meaning as would be commonly understood or used by one of ordinary skill in the art encompassed by this technology and methodologies.

Texts and references mentioned herein are incorporated in their entirety, including U.S. Provisional Patent Application Ser. No. 62/645,652 filed on Mar. 20, 2018.

In an embodiment, methods for measuring cellular responses to differential stimuli using optical and/or fluidic forces, wherein such methods comprise receiving a selection of an initial samples comprising biological cells treated with varying known levels of stimuli or analyte, performing optical force-based measurements on the samples, developing a response metric (RM) to describe the cellular response to the stimuli based on one or more optical or fluidic force-based parameters are provided. In certain embodiments the methods as disclosed herein may be computer-implemented.

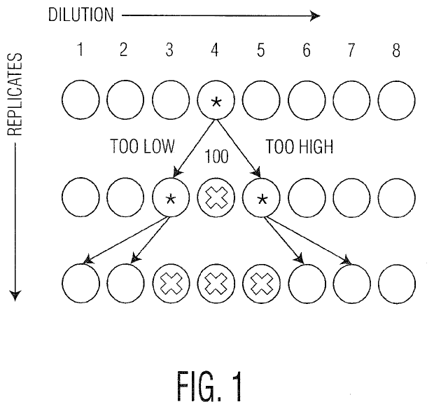

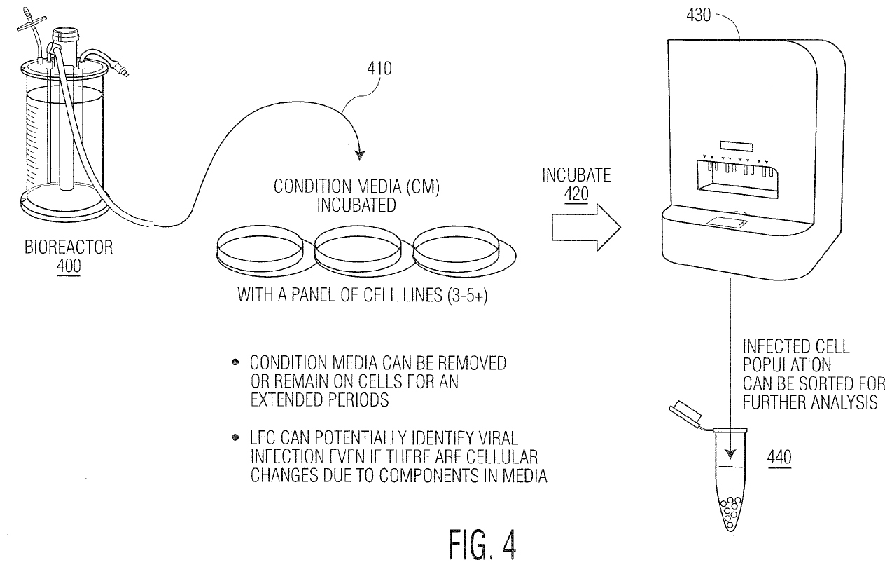

As illustrated in FIG. 1, an intelligent algorithm (100) is designed to be used for reading (detecting), analyzing and predicting cellular changes, such as, but not limited to, cytopathic effect (CPE) (for example % CPE for viral, bacterial, or toxin effects. Alternatively any LFC measured parameter including but not limited to effective refractive index or size normalized velocity could be used to describe cellular changes instead of % CPE) of samples contained in a multi-well plate (96-well is a preferred embodiment, but “well plate” may hereafter be understood to mean any well plate, including but not limited to a well plate containing any number of wells, or pattern(s), or a vessel (see e.g., FIG. 4)). Algorithmic software, in one embodiment, initiates instrumental analysis and detection of cellular change, i.e. % CPE, in the starting well position. In aspects, this starting position can be chosen by the user based on experience or other pre-programmed homing coordinates. The algorithm, in embodiments, will subsequently automatically select a well with either a higher or lower dilution based on the observed data, the data trend, and/or the experiment layout previously loaded into the software. Specifically, sampling begins at an intermediate dilution or untreated control based upon user input or prior knowledge. The next sample to be analyzed is chosen based upon the quantitative results of the initial sample. More specifically, for infectivity measurements, this could refer to the % CPE. Thus, if the % CPE is higher than the target infectivity value (e.g., 50%), then the next sample analyzed would be one containing a larger dilution factor (e.g., lower concentration of analyte, such as virus or neutralizing agent). The size of the interval moved depends upon the magnitude of the measurement. For example, a CPE value near the maximum (100%) might warrant moving two to three dilutions lower, while a CPE value closer to desired value (50%) would require moving only one (1) dilution lower. Conversely, if the initial measurement is lower than a target value, the next sample measured will be a smaller dilution factor (higher concentration of analyte), and the magnitude of the interval would again be based upon the magnitude of the measurement. The subsequent dilutions sampled are selected in a similar fashion, until the target dilution(s) are identified or the plate (in part or in whole) has been analyzed. Thereafter, replicates at the same dilution are sampled until an accurate measurement of the infectivity can be determined. If there is limited prior knowledge or understanding of the level of infectivity or analyte expected, sampling can begin in the middle and proceed in an automated fashion based upon the measurements until the target infectivity has been identified. This can ultimately result in a reduced number of dilutions and/or replicates required to accurately measure the infectivity of the sample. Thus, the novel methodologies provided herein reduce the number of sample dilutions required, as compared to the number required by traditional neutralization assays, and also decrease the time required for well plate analysis by the application of an intelligent algorithm and the larger dynamic range afforded by the use of optical force-based technologies such as laser force cytology (LFC). In an embodiment, the optical force-based technology utilized comprises laser force cytology (LFC), however any other optical force-based technologies could be used with the invention as described herein, including but not limited to optical chromatography, cross-type optical chromatography, laser separation, orthogonal laser separation, optical tweezers, optical trapping, holographic optical trapping, optical manipulation, and laser radiation pressure.

In an alternative embodiment, the IA (100) could be set to automatically search for certain conditions, including various time points, dilutions, or reagent variations at one or more sampling timepoints. Accordingly, the IA (100) could monitor the lowest dilution, extrapolate and predict concentration and sampling requirements, and calculate an estimate for the next analysis using optical force-measurements (i.e. LFC) and enable calculation of the Infection Metric/mL (“IM”) (120). As used herein, the term Infection Metric (“IM”) or Response Metric (“RM”) refers to a specific parameters or values that take into account cell counts, velocity (including changes in velocity and position during flight time), optical force, size, shape, aspect ratio, eccentricity, deformability, orientation, rotation (frequency and position), refractive index, volume, roughness, cellular complexity, contrast based image measurements (e.g., spatial frequency, intensity variations in time or space), 3-D cell images or slices, laser scatter, fluorescence, Raman or other spectroscopic measurement and any combination of or other measurement made with respect to the cells or population that reflects the level of cellular changes or viral/bacterial infectivity in a sample. In an embodiment, a device such as Radiance™ (a laser force cytology instrument available from LumaCyte™ (Charlottesville, Va., USA) is used for conducting optical force-based measurements, however as would be evident to one skilled in the art, other devices and methods capable of optical force measurement including LFC would be suitable for use in connection with this invention. (For clarity infection metric (IM) and response metric (RM) can be used interchangeably depending on the type of measurement being made.)

One IA embodiment, labeled as (120) in FIG. 1, is developed by measuring a number of samples at various levels of infectivity in order to determine how Radiance™specific parameters that are measured change upon infection. As indicated in FIG. 1, the LFC instrument (“Radiance™”)-associated software automatically calculates (120) for each sample when these parameters are measured on a per cell basis. (120) can be equated to traditional TCID50/mL, pfu/mL, multiplicity of infection (MOI) or other known infection values but also contains additional quantitative information about the cell population. The per-cell multi-parameter analysis yields data that can detect much more sensitive shifts in or differences between cell populations and viral strain infectivity rates. The application of (120) to various cell lines and viral strains can also be, in the alternative, normalized to correlate variances and or similarities between infection models and sera from vaccinated or non-vaccinated samples where levels of drug or vaccine-induced antibody in the blood can be examined for effects on cells. Moreover, results from bacterial or viral infection of cells and can be further compared between and across various studies for trends and cell population comparisons.

Interpolation between dilutions and replicates using a quantitative measurement of % CPE (140) can be made by adjusting (100) to extrapolate data from analyzed wells to determine interstitial log or exponential data points for highly accurate and sensitive analysis that is directly correlative to observed phenomena. This predictive algorithmic determination can inform the user of desired dilution or replicate stratagem for future experimentation and sample manipulation.

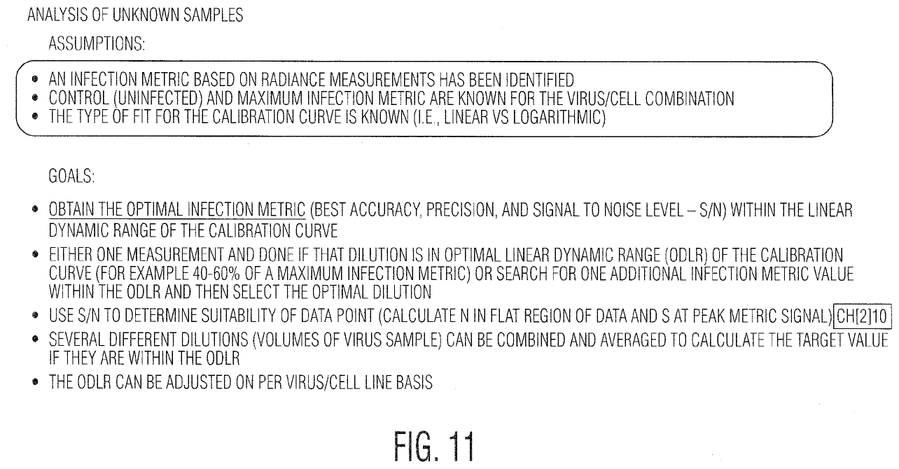

FIGS. 11, 12, and 13 provide additional details regarding the details of an example IA for measuring infectivity. Although this embodiment describes the calculation of infectious viral titer (infectivity) based on Radiance measurements, the algorithm could be applied to other systems in a similar way. FIG. 11 lists the Assumption and Goals for this particular embodiment. Specifically, the assumptions include that an infection metric based on Radiance measurements has been identified, that control (uninfected) and maximum values for the infection metric are known for the virus/cell combination, and the type of fit for the calibration curve is known. The goals of the IA are to obtain a value or values of the RIM that maximize the accuracy, precision, and signal to noise ratio of the infectious viral titer or infectivity. There should be a range of values for the RIM that ensure this, which are calculated based on previous data used to create the calibration curve. The range is terms the optimal linear dynamic range (OLDR) for the calibration curve and may be adjusted on a per virus/cell line basis. In addition, it could be possible that multiple values are measured within the OLDR and are then all used to calculate the resultant infectious viral titer or infectivity.

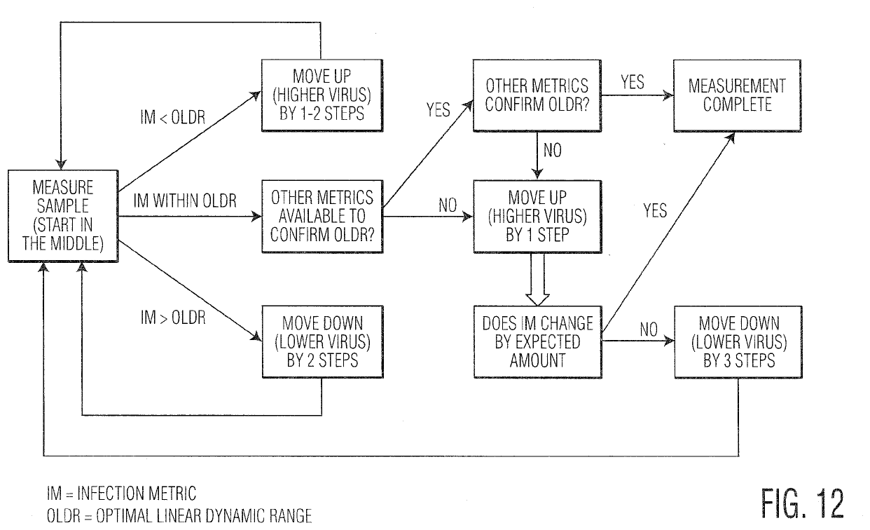

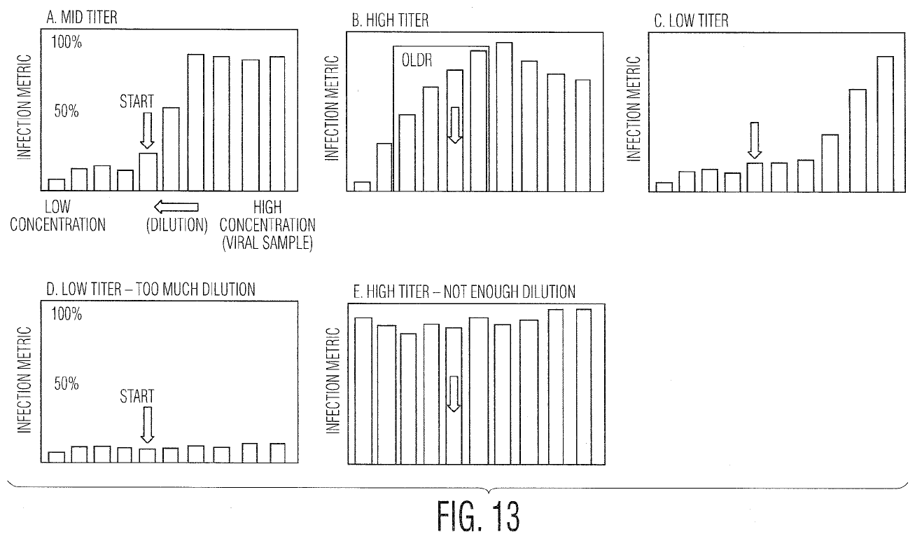

FIG. 12 shows a flowchart that describes the example algorithm. The first step is to measure a sample, the first of which is generally within the middle of the range of dilution values. If the value of the RIM is outside the OLDR, then a different well is sampled, moving to a higher concentration of virus (analyte) if the IM is too low, and moving to a lower concentration of virus (analyte) if the IM is too high. Once the value of the IM is within the OLDR, a check is made to confirm whether or not the sample is truly within the OLDR. The reason for this is illustrated in FIG. 13, which shows several example graphs showing the variation of the IM as the concentration of the virus (analyte) changes. In some cases (shown in A. Mid titer), the values of the IM plateau for high concentrations of analyte, in which case there would be less potential for confusion as to whether or not a single measured value is actually within in the OLDR. In other cases (shown in B. High titer), the value for the IM at very high concentrations which are outside the OLDR can be the same or even less than values that are actually within the OLDR. Thus, a check must be made as part of the example algorithm in FIG. 12 to ensure values are within the OLDR. The first part of the check is to see whether or not other characteristics and measurements of the sample that are not necessarily part of the IM can be used to determine whether or not the sample is truly within the OLDR. This could be based on prior knowledge related to the biology of the system as well as potentially other measurements made in the LFC system. If other metrics are available to confirm the OLDR, then the algorithm proceeds according to the results of that test. If the other metrics confirm the OLDR, then the measurement is complete and the titer (infectivity) can be calculated. If the other metrics cannot confirm the OLDR, then the IM is measured for the next highest concentration of virus (analyte). The same step is performed if there are no other metrics available to confirm the OLDR. Based on the IM of the higher virus concentration, the algorithm proceeds accordingly. If the IM changes by an expected amount based on the previous knowledge of the calibration curve, then the value is confirmed to be truly in the OLDR and the measurement is complete. If not, then the value is outside the OLDR and likely too high, so the next sample measured is 3 steps lower in virus concentration.

Additional cases are shown in FIG. 13 describing potential trends or cases of the variation in the IM with changes in virus (analyte) concentration. In addition to the two cases already described, 13C. shows a low initial concentration of virus such that fewer values at the sampled volumes are within the OLDR, while 13D. shows an initial concentration so low that all the dilutions measured are outside the OLDR. Finally, 13E illustrates an initial concentration that is so high that all the values are also outside the OLDR.

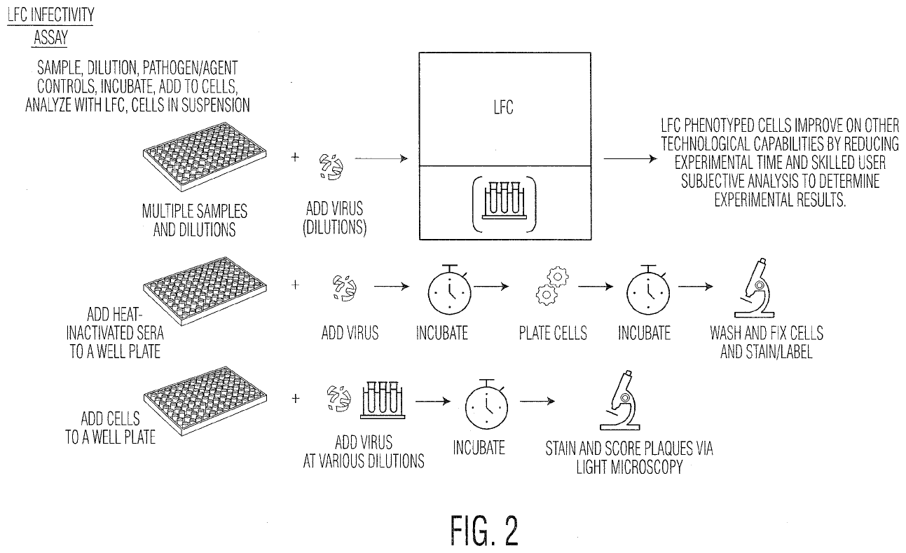

The schematic in FIG. 2 illustrates previously patented laser analysis and sorting technology (“Radiance™”), incorporated herein by reference, for background and preferred embodiment application where samples are derived from a neutralization assay containing multiple patient serum-virus dilutions and cells of choice and are analyzed by LFC (200). For neutralization assays, serum and virus are incubated in a well plate for a period of time before combination with the cells and subsequent incubation. After the incubation period, samples are analyzed by Radiance™ in order to determine infectivity values including calculation of (120). Traditional neutralization assays inherently require the use of adherent cells for assay performance. As viruses infect many mammalian and insect cell lines which require growth and infection while in suspension (physiological demands), this can limit the models used for neutralization assay studies. Radiance™ enables the analysis of suspension cells for neutralization and other infectivity assays by not requiring flat well plate or adherent cells for the technology to process and measure samples. The use of suspension cells (160) further allows for potentially more uniform infection and sampling of the same well over time (e.g., periodic sampling). In another embodiment, cells can be suspended in an alginate, gelatin or other similar semi-solid suspension prior to sampling in order to reduce adherence to tissue culture plate surfaces during extended incubation times and/or provide a physical environment more representative of in vivo conditions (180). The potential use of a suspension matrix further enables dilute cells to be infected in relative isolation from potentially interfering contact signals from other cells and enables more accurate physiological relevance for infection models than is currently embodied by the prior art. Moreover, Radiance™ and IA (100) permit a percent neutralization to be calculated for virus or other pathogens. In an embodiment, Radiance™ and IA (100) can be utilized for automatically analyzing and scoring CPE or plaque formation in TCID50 or plaque assays (220) as well as for AAT whereby infected cells are sampled periodically to detect the presence of bacteria, virus or another pathogen. In this case, the virus or other analyte would not be incubated with neutralizing serum but instead combined directly with the cells.

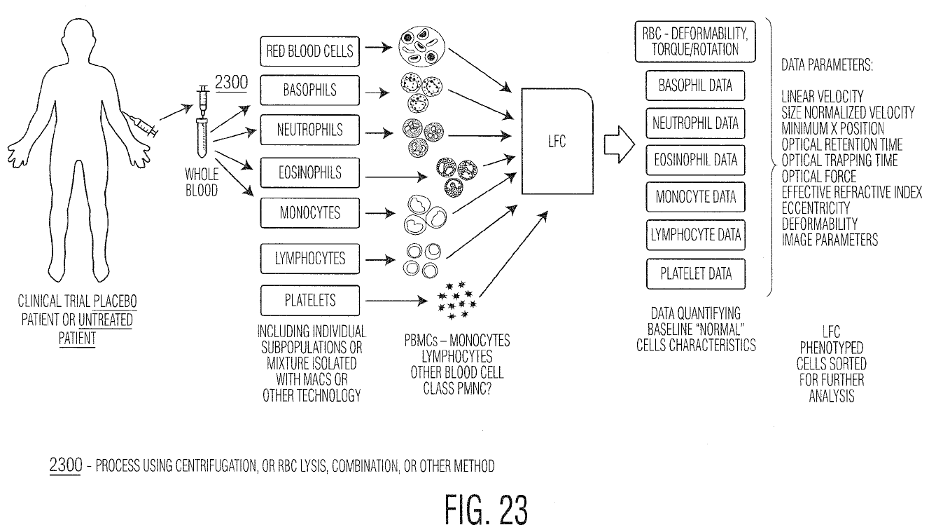

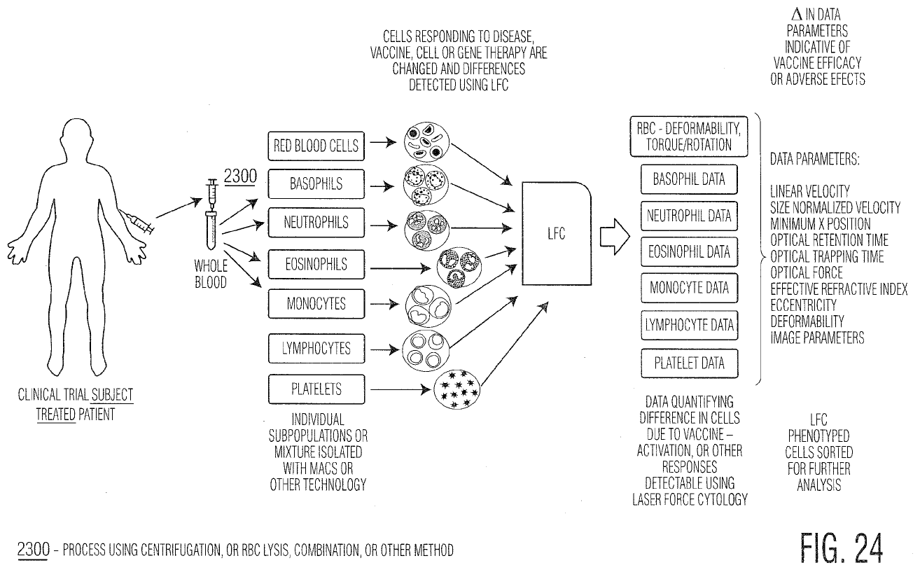

Measurement of cellular changes is possible using LFC for any type of cell or particle for changes due to viral, bacterial, protozoan, or fungal infection, cell differentiation, necrosis, apoptosis, aging, maturation, malignancy (cancerous tissue, cells, material circulating or not), exosomes, antibodies, proteins, or small molecules. Cells within animal or plant systems can behave as sentinel cells in that they respond and change in ways detectable using LFC. Changes in the biophysical, biochemical, or other properties of cells or other biological particles can change due to various external or internal changes or insults such as those described above. The ability of LFC to detect and measure such subtle changes (Response Metric (RM)) enables it to be a tool for biomarker discovery and identification, for particulates in animal, plant, protozoan, or fungal systems. These biomarkers are important for detecting new or changing cellular states either related to disease or biological process. FIGS. 23 and 24 provide examples of these concepts wherein a human patient has a disease or is given a treatment (chemical, vaccine, cell or gene therapy for example but not limited to) and their blood cells (red blood cells, white blood cells, platelets—separated or not), exosomes, or other cells or biological components change in response to the disease or treatment (for treated patients). LFC can detect these changes, which can then form the basis of the biomarker for future monitoring.

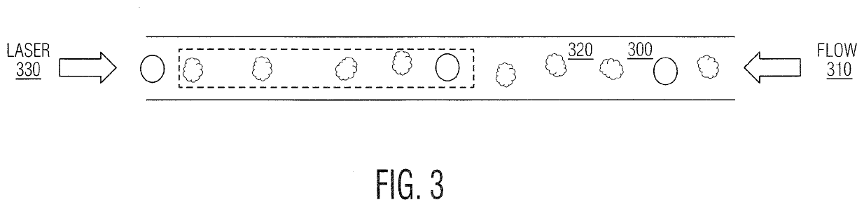

The use of one or more types and/or sizes of internal calibration objects (beads or particles) (240) may be used, as in FIG. 3, to increase the confidence that experimental samples are behaving in a consistent manner. Concurrent calibration can yield enhanced titering performance by monitoring system performance throughout plate analysis, reduce error and standard deviation between samples, enable the data to be rejected or accepted according to experimental parameters and/or normalized to ensure inter and/or intra experimental consistency (whether fixed, freeze-dried or artificial). Calibration objects could, in certain embodiments, be used at the beginning of every row, or once on the plate, depending on the nature of the samples, and the desired level of calibration required. The current invention describes measurements such as optical force, optical torque, optical dynamics, effective refractive index, size, shape, or related measurements of calibration objects alone or mixed in with cells wherein said objects are polymer, glass, metallic, alloy, biologic, lipid, vesicles, or cell (live or fixed) based. Calibration objects should have properties related to the particles of interest, yet not interfering with data collection on samples of interest. Calibration objects could be used alone, mixed with a sample of interest, mixed with different types of calibration objects, or any combination of the three. Optical force and other measurements as described above can be used to calibrate, verify, or enhance the performance of the system as well as normalize or compare data across different systems.

In an embodiment, methods for generating calibration curves based on cellular response to varying concentrations of treatments and then using such curves for predicting characteristics of a sample of an unknown level, are provided. Such methods comprise the steps of adding treatments and incubating sample cells, analyzing by optical force-based measurements a plurality of samples having cells, and a known range of treatments to determine a response metric, determining optimal response metric and time based on trend with dilution, and using generated data to predict future samples.

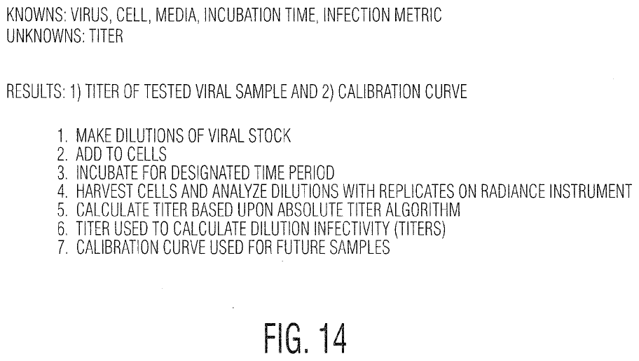

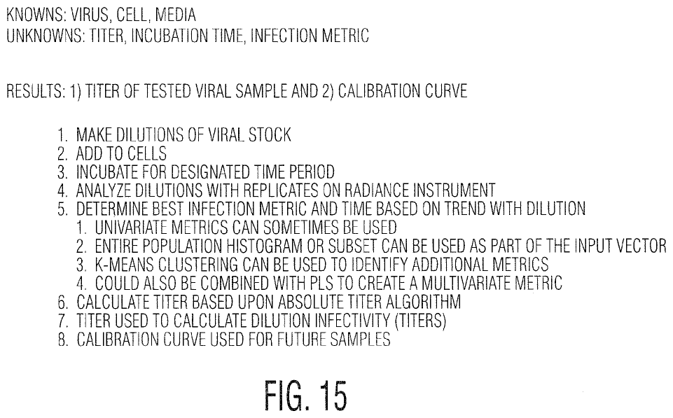

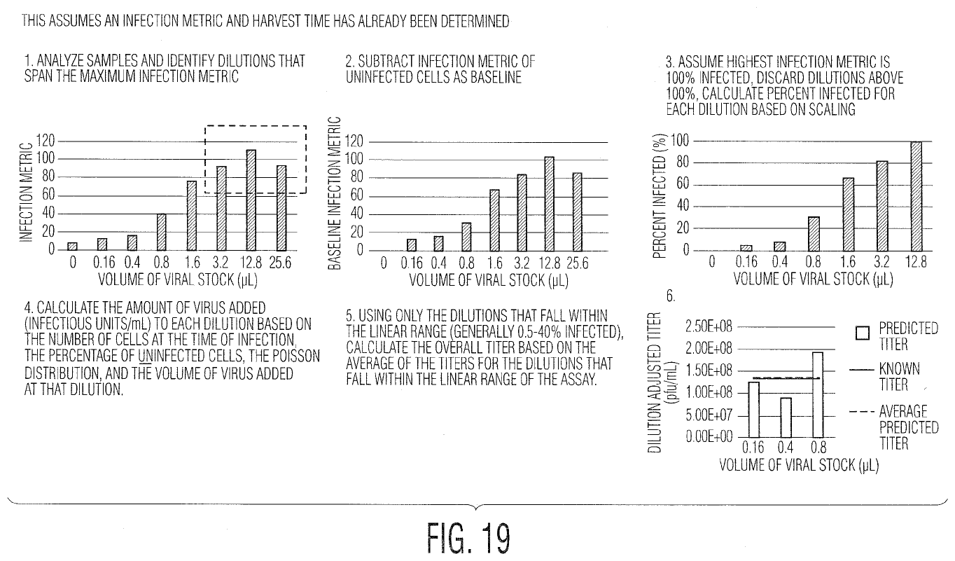

Two embodiments of the steps required to create a representative calibration curve are shown in FIGS. 14 and 15. FIG. 14 describes the process for calculating a titer and creating a calibration curve from a known or well-understood viral sample with a sample of unknown titer. Well-understood means that both the IM and incubation time for calculating the titer has been established based on previous experiments. In this case, dilutions of unknown viral stock are made and added to cells before incubation for the designated period of time. Then the cells are harvested and analyzed using Radiance™ or a similar instrument capable of making optical force based measurements. The titer (infectivity) is then calculated based on the absolute titer/infectivity algorithm described in FIG. 19. Once the titer is calculated, the calibration curve can also be developed by using the titer value determined to calculate the viral concentration at each of the dilutions. This calibration curve can then be used for the measurement of future unknown samples.

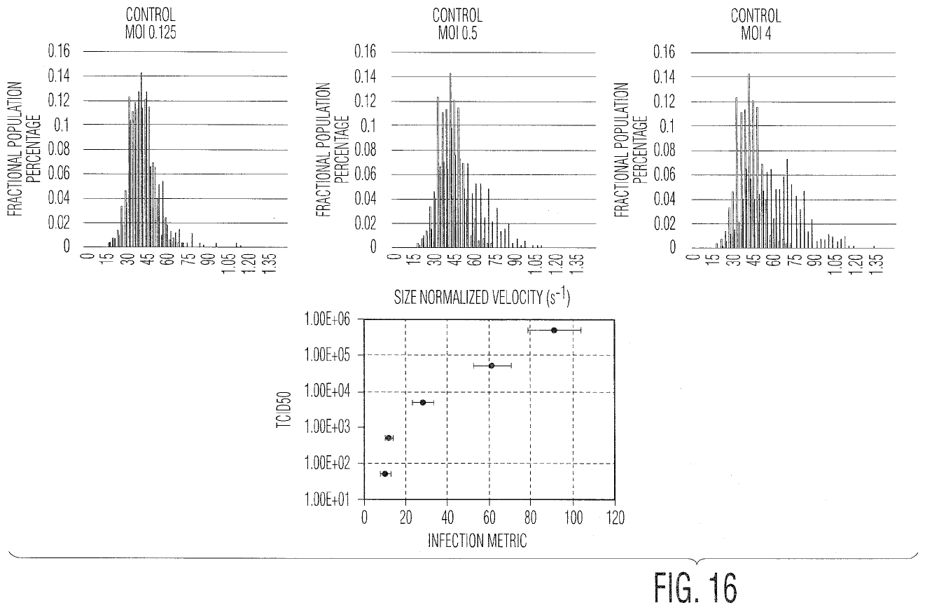

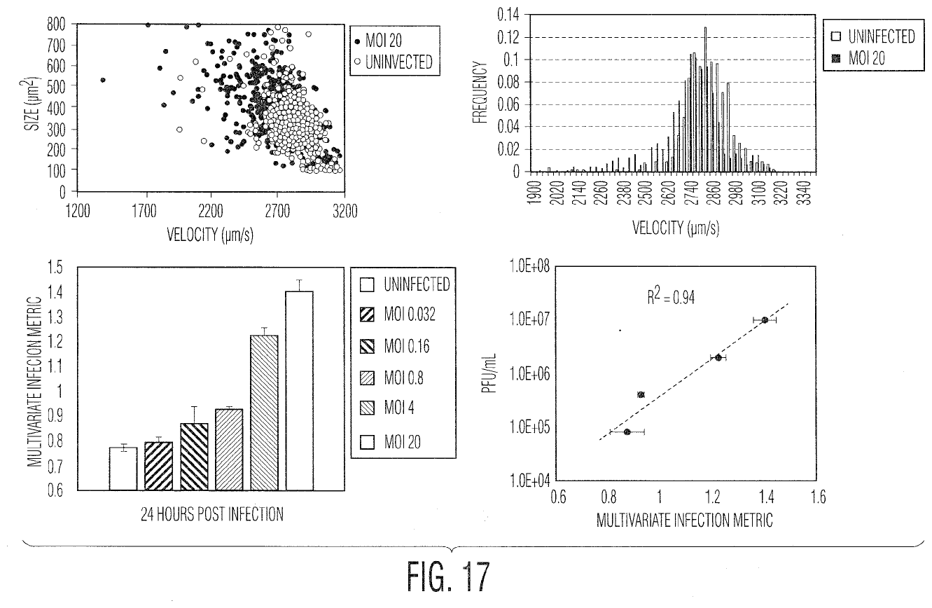

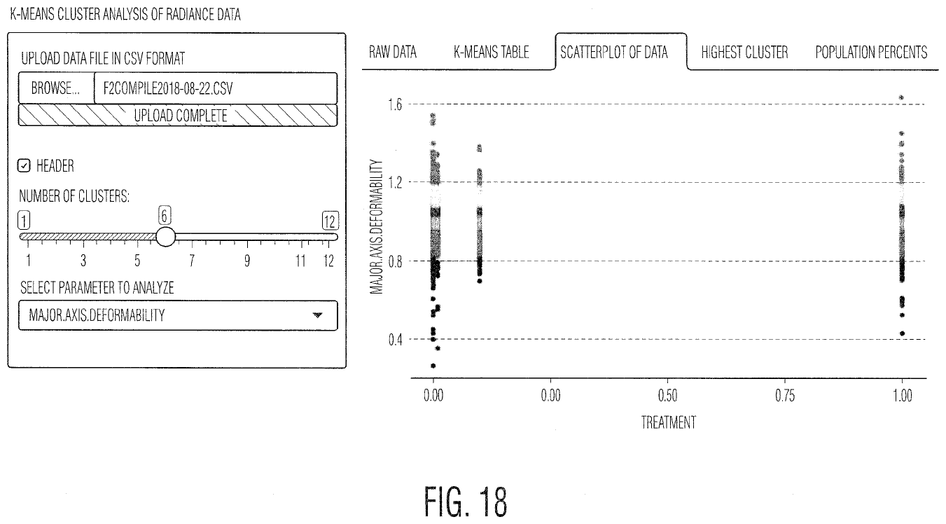

FIG. 15 describes the process for calculating a titer and creating a calibration curve from an unknown or not well understood viral system with a sample of unknown titer. In this case, the virus and cell line are known, but the IM and incubation time are unknown. Thus, experiments must be conducted in order to determine both the incubation time post infection, as well as which LFC parameters are used to calculate the infection metric. There are several ways to generate these metrics, as described in FIGS. 15-18, though the overall goal, independent of which parameters are used to calculate the IM, is to develop a parameter (or a set of parameters) that correlate well with the infectious viral titer over as wide a range of viral concentrations as possible. An example of this is illustrated in FIG. 16, showing the histogram of one of the LFC parameters, size normalized velocity, and how it changes with respect to the amount of viruses added (MOI). In this case, Vero cells have been infected with vesicular stomatitis virus (VSV). As shown, the size normalized velocity increases as the MOI increases, ranging from MOI 0.125 in the first histogram to MOI 4.0 in the last histogram. The size normalized velocity, coupled with the standard deviation of the velocity, was used to develop an IM that correlates strongly with the MOI and thus viral concentration. FIG. 17 shows data from another viral system, human adenovirus 5 (Ad5) infecting human embryonic kidney (HEK 293) cells. It also illustrates another technique for developing the IM, partial least squares (PLS) analysis. In this case, as many parameters as needed can be added to the PLS calculation in order to develop a multivariate IM. The inputs for the PLS model can be population wide statistics, such as the average, standard deviation, or median for any parameter measured by the LFC instrument, but also more complex inputs, such as a population histogram for a particular parameter, such as velocity. The bins of this histogram can be defined simply based on a standard distance between the bins, or can be adjusted based on a clustering algorithm, such as K-means clustering, shown in FIG. 18. In the case of K-means clustering, the number of bins as well as the parameter used can be defined. Also, in general, either the entire population or only a portion thereof can be used to define the population histogram.

FIG. 19 describes one particular method for calculating the titer (infectivity) of an unknown sample when the infection metric and incubation time is already known. As described in FIG. 15, cells are infected with different dilutions of virus and then the infection metric is calculated for each sample as it is analyzed after the designated post-infection incubation period. At an above a certain concentration of virus, essentially all of the cells should become infected during the first round of infection. Multiple distributions have been developed to describe viral infection, but one specific example that is often used is the Poisson distribution. In general, the infection metric will have a maximum or plateau above a given viral concentration. Thus, the first step when analyzing an unknown sample is to identify the maximum infection metric as well as when the infection metric starts to decrease below that maximum, which should occur in a known fashion based upon the assumed distribution for viral infection. By understanding this distribution as well as the number of cells and volume of virus added, the number of infectious units of virus can be added. Once the point of maximum infection metric is determined, in the specific example shown this occurs at MOI 4, the next step is to subtract the baseline infection metric of the uninfected control cells. It is assumed that 100% of the cells are infected as the point of maximum infection, which allows for the calculation of the percent of cells infected at the lower virus concentrations by scaling the infection metric in a linear fashion. The next step is to calculate the amount of virus added in infectious units/mL at each dilution, based on the number of cells at the time of infection, the percentage of uninfected cells at each dilution, the Poisson distribution (though other distributions could be used), and the volume of virus added at that dilution. The equation for this relationship is:

$$Titer ( \frac{Infectious Units}{mL} ) = - ln P ( 0 ) x n / v$$

Where P(0) is the fraction of uninfected cells, n is the number of cells at the time of infection, and v is the volume of the original viral stock added (mL). Based on the Poisson distribution, it is assumed that:

$$MOI ( \frac{Infectious Units}{cell} ) = - ln P ( 0 )$$

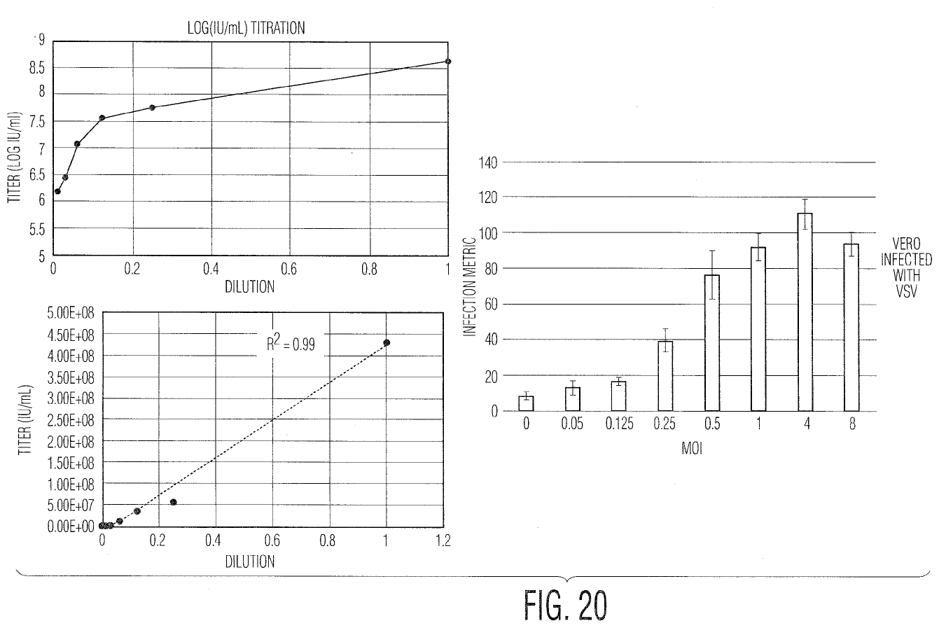

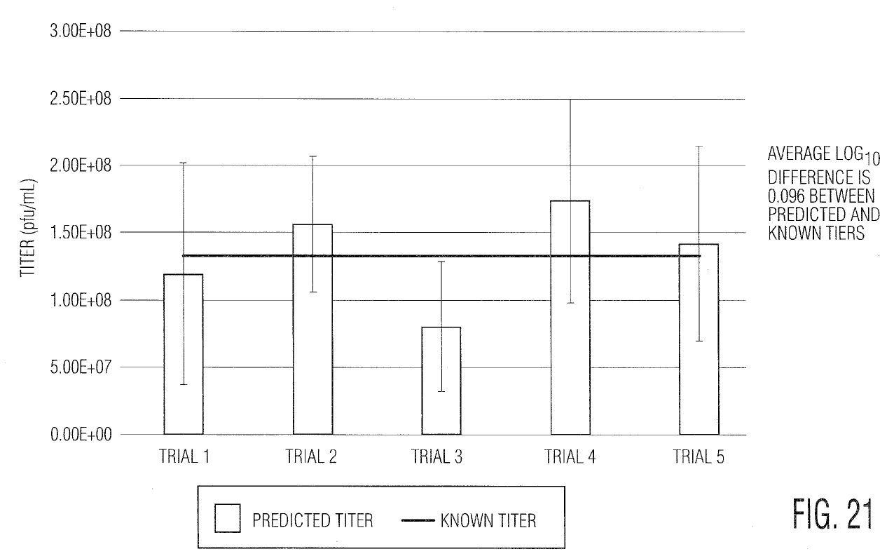

As part of the next step, the dilutions that fall within the optimal range for the calculation are determined. Generally, this is between 0.5% and 40% infected. Once these dilutions are determined, the overall titer (infectious units/mL) can be calculated based on the average titer from the 2-3 dilutions within the OLDR.
Specific data showing the relationship between the dilution and titer is shown in FIGS. 20 and 21. FIG. 20 shows the correlation between dilution and titer on both a linear and logarithmic scale, as well as the relationship between the MOI and infection metric for this particular data set. FIG. 21 shows the absolute titer/infectivity predicted from 5 independent experiments based on this calculation. The average difference between the known and predicted titers is 0.096 log₁₀.

Analysis of infectivity based on optical force-measurements is also possible in multiple formats on devices such as Radiance™. Forms of sample housing include but are not limited to well plates of various well plate number or size configurations (flat or U-bottom) such as 6, 12, 24, 48, or 96 well plates, patterned surfaces with wells, spaces, grooves, or other raised or indented features for cell culture, flow or suspension, droplets of one or multiple cells on, in or independent of well plate or microfluid structures, other vessels such as culture dishes, flasks, beakers, bioreactors or tubes which can house larger volumes of samples. The ability to alter the format of sample preparation enables the user to utilize any number of multiple experimental designs including varying sample size, dosing/dilutions and/or magnitude of samples analyzed in one preparation.

As is known to those skilled in the art, one serious concern associated with the manufacture of biological products such as vaccines and cell and gene therapy products, is the inadvertent introduction of adventitious agents (endogenous or exogenous). The use of optical force-based measurements, such as those obtained using LFC to detect adventitious agents (AA) in bioreactor condition media or other fluids used in biomanufacturing, is an important capability of the novel methodologies described herein. The methods of the present invention enable the critical assessment of quality and prevention of bacteria, viruses, or other replicating/living contaminants from jeopardizing the production of drug substances. The ultimate goal of advanced AAT using LFC is to thwart the possible inclusion in a drug product that could lead to potential infection of patients. The overall process for using LFC for measuring viral infectivity in biomanufacturing is shown in FIG. 4 where condition media (CM) from a bioreactor or other manufacturing process is mixed with cells growing in suspension or adherent culture and incubated for a shorter period than current methods which currently take 14 days or more under FDA guidelines. The same cells are monitored using blank samples as controls. The amount of time the cells are exposed to the conditioned media can be adjusted as part of the assay optimization.

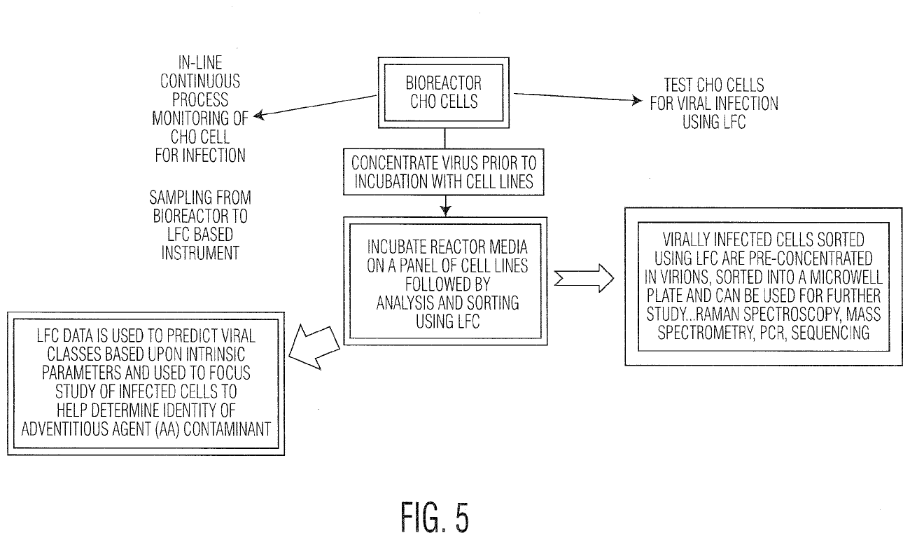

In an embodiment, the first line of defense when using LFC to monitor for AA is using CHO or another cell line used for bioproduction directly as a responsive cell that can be measured using LFC. While not all viruses cause cytopathic effects in CHO cells (and other production cell lines), many do, and this forms the basis for real-time monitoring of changes in CHO cells during production. Deviations in variables measured using LFC can be used as indicators of potential contamination by AA. This is shown in FIG. 5 where the overall strategy for AAT using Radiance™/LFC is given. CHO cells used in production are constantly monitored by a sampling system that removes cells and introduces them to Radiance™ for LFC analysis to gauge changes in their intrinsic properties as a way to monitor for AA. CPE may be visible if AA are present and this differs from any changes in LFC measured variables used to monitor protein production. Samples could also be removed from the bioreactor and run separately in Radiance™ using LFC as opposed to on-line analysis. Condition media (CM) can be removed and incubated with cells with or without concentration (e.g., centrifugation to concentrate potential AA). After an incubation period or throughout the incubation period, cells can be monitored for signs of AA. Radiance™/LFC can sort out potentially infected cells and collect them for analysis using other methods including spectroscopic (fluorescence, Raman, or other), polymerase chain reaction (PCR), next generation sequencing (NGS), mass spectrometry (MS), cytometry (flow, fluorescence, mass, or image) or other methods.

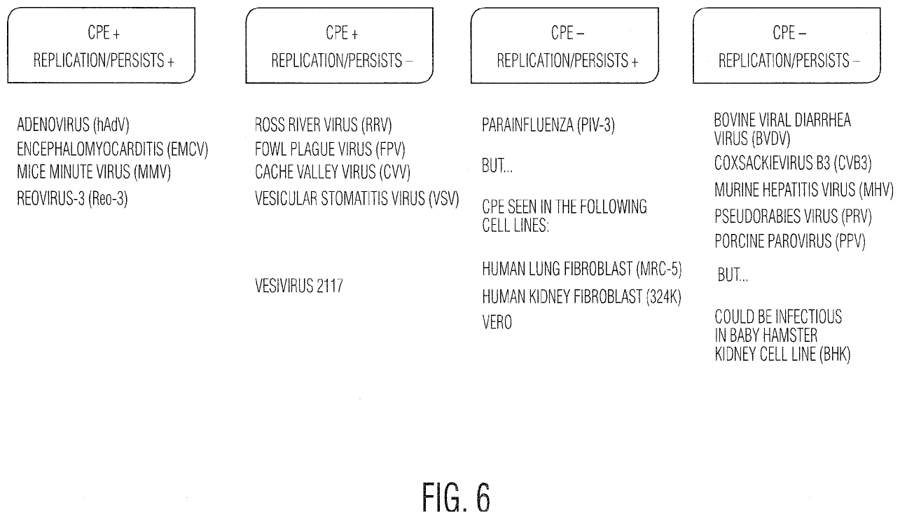

For those viruses that do not cause cytopathic effects in CHO cells, other cell lines can be used for detection. FIG. 6 shows a partial list of viruses and classifies them according to cytopathic effect and replication. This indicates that four cell lines can provide decent coverage of potential viruses: Vero cells, baby hamster kidney cells (BHK), MRC-5 cells, and Human kidney fibroblast (324K) cells. The panel is not limited to these four cell lines and other existing cell lines can be used, as well as newly developed cell lines modified for specific susceptibility.

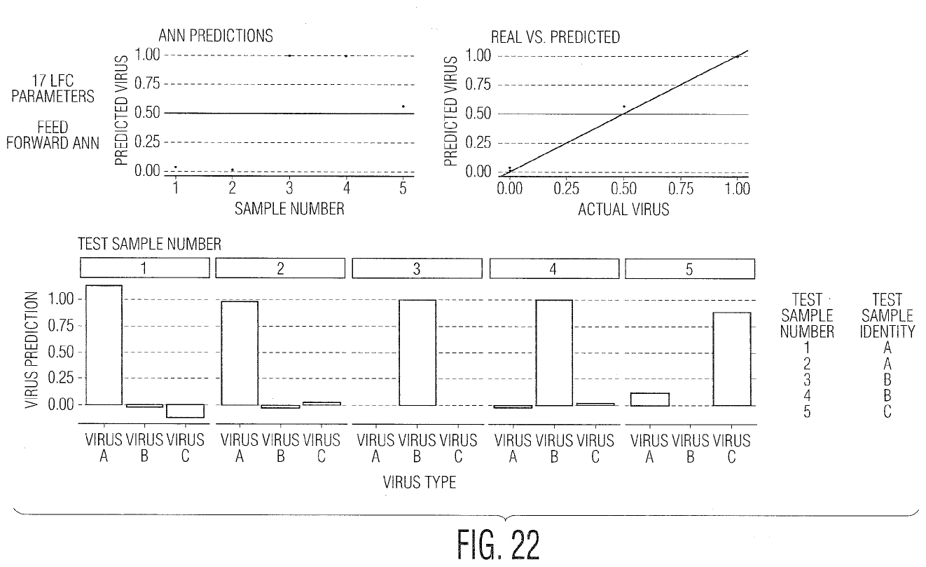

In an additional embodiment, the methods described herein may be used to to classify viruses or other AA based on a specific pattern of data. Several methods could be used for this, including artificial neural networks (ANN), pattern recognition, or other methods of predictive analytics. A specific data example of this using LFC data is shown in FIG. 22. Here, an ANN is used to classify test samples as one of three potential viruses using approximately 17 LFC parameters as the input.

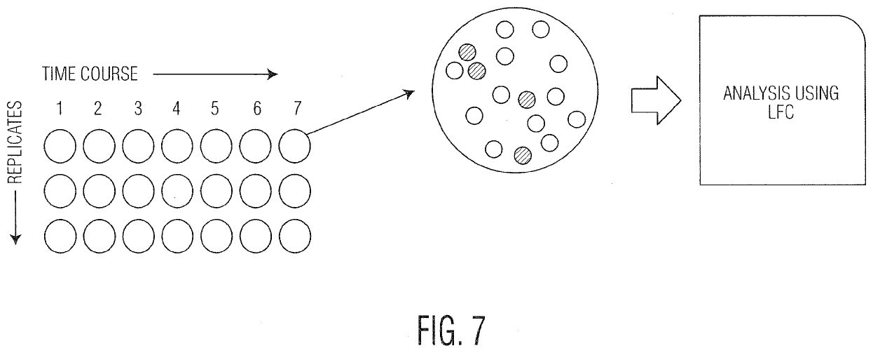

In certain embodiments, to speed analysis, multiple cell lines can be run simultaneously as in vitro sentinel cell lines with condition media (CM) or another analyte. In certain embodiments, sentinel cells are cells that are susceptible to the condition (viral, bacterial, mycoplasma, infection, or other AA) being monitored or detected and their response can be measured using LFC. FIG. 7 shows a multiplexed assay using multiple in vitro sentinel cell lines in each well or biosampling system. The ability to differentiate the cells in Radiance™/LFC by parameter space or using other tags, fluorescence, visual brighfield microscopic identification, or others means would greatly increase throughput by allowing the cells to be incubated together and run at the same time. Cells engineered to have different parameters in Radiance™/LFC so they will not be confused with one another can be used to multiplex the assays. Methods to multiplex by modifying the cells to have different properties include but are not limited to: Fluorescence based—green fluorescent protein (GFP), red fluorescent protein (RFP), yellow fluorescent protein (YFP) and other genetic modifications incorporated into macrophage line or other cell lines so one can determine which one is reporting presence of cytopathic or other effect due to AA. Cells analyzed using LFC can also be labelled with, by way of example only, stain, dye, antibody conjugated bead labels, affinity bound beads or molecules, nano-particles (Au, Ag, Pt, glass, diamond, polymer, or other materials). Nanoparticles could have different shapes (spherical, tetrahedral, icosahedral, rod or cube shaped, and others) and size to accomplish two objectives: 1) varied entry into cells, and 2) changing the optical force measurable using LFC.

In certain embodiments, nanoparticles may be incubated with the cells and uptake would happen as normal for the cell type or alternatively nanoparticle uptake could be augmented chemically or physically (such as by electroporation or facilitated by liposomes) to enhance nanoparticle uptake percentages. Cells would be incubated with nanoparticles and a virus to be tested and an increased differential of viral uptake into cells would lead to a larger differential in optical forces measured using LFC, thus improving viral detection sensitivity. In alternate embodiments, nanoparticles may be incubated with the virus prior to exposure to the cells.

In additional alternate embodiments, macrophages that engulf a specified number of beads would have different properties in LFC but would still report the presence of AA. Additionally, only specific portions of the cell could be analyzed, such as the nucleus, mitochondria, or other organelles. This could be used to enhance the performance not only AA but also other cell-based assays including infectivity.

In aspects, cells may be genetically engineered to have different viral, bacterial, fungal, or other AA susceptibility for use as in vitro sentinel cells, in an embodiment, in the panel used with Radiance™/LFC would allow a tailored approach to AA detection. Incorporating or eliminating certain genes into or from the cell line may make the cell line more permissive to infection with a particular class of viruses, bacteria, or other AA, thus affording rapid detection with selectivity of pathogen type. This combined with the broad viral identification possible using LFC will allow better identification of viral, bacterial, or other type of AA.

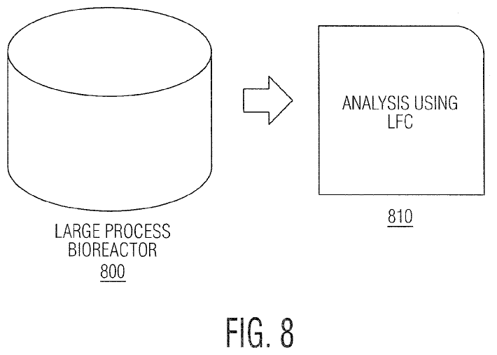

The novel methods described herein demonstrate that AAT could occur directly on cells removed from the production bioreactor (800) through analysis immediately using LFC/Radiance™ (810) as shown in FIG. 8. For AA that do not produce CPE or other effects in the production cell line (CHO or others), additional suspension cell lines can be used in mini analytical bioreactors (910) to spur growth and infection with any AA present in the production bioreactor.

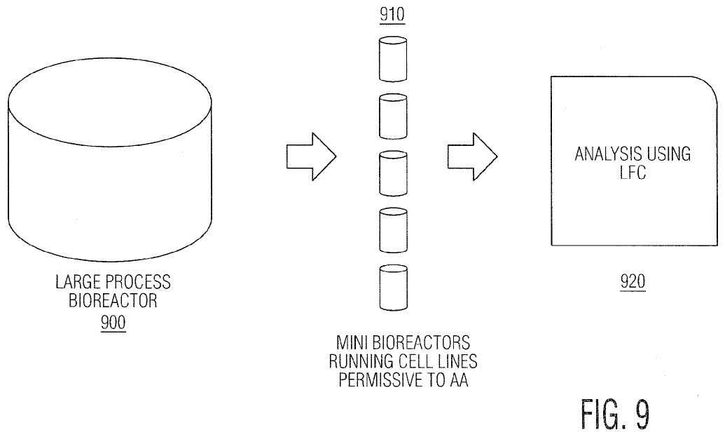

Cell lines grown in mini bioreactors (910) for subsequent sampling with, for example, Radiance™ (920) can be used to test CM for AA, as shown in FIG. 9. Samples of CM are pumped into mini bioreactors from a large process bioreactor (900) that can then be sampled using LFC technology (920) (e.g., Radiance™) periodically to ascertain if adventitious agents are present. Multiple bioreactors can be used to sample at different time points in the production process if needed. The mini bioreactor(s) would, in aspects, have optical windows for spectroscopic analysis of cell lines for signs of infection that could be used to provide identification of virus infection or mycoplasma, or prions, or bacterial, fungal, or protozoan infection.

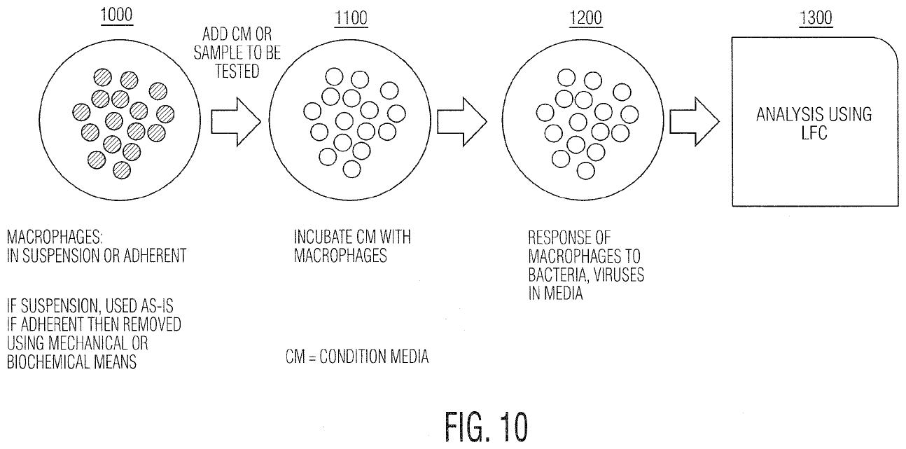

FIG. 10 shows the use of macrophage cells (white blood cells that engulf foreign material including viruses, bacteria, vegetative spores, and almost any other material), in this example as in vitro sentinel cells, for the detection of AA present in CM. The macrophages respond to the presence of foreign materials in unique ways detectable via LFC and can also engulf the foreign material (virus, viral inclusion bodies, bacterial spores or vegetative cells, exosomes, or any other biological material) thus increasing their refractive index by concentrating AA inside their membranes as they engulf them. This serves to increase the LFC response to AA and also to make the macrophages a convenient and detectable container or vehicle for LFC to sort and deliver preconcentrated AA to other techniques for further analysis. It will be important in this application to exclude the bioproduction cells (CHO or others) so they are not engulfed by the macrophages, influencing the assay outcome. Although presumably the CHO cells² would generally not be engulfed as they are the same size or larger than the macrophages³. Alternative macrophage activation (known activators such as plate binding, plate composition, media additives, addition of biomolecules including lipopolysaccharides (LPS), bacterial or viral proteins, among others) could be used to selectively control phagocytic activity or phenotypic state including changes in gene or protein expression.

Specificity in viral, bacterial, or other organism detection is made possible through the use of the many parameters that LFC/Radiance™ measures, including size, velocity (related to optical force), size normalized velocity, cellular volume, effective refractive index, eccentricity, deformability, cell granularity, rotation, orientation, optical complexity, membrane greyscale, or other parameters measured using LFC/Radiance™. This represents the use of multivariate parameter space including images to define classes of viruses or other organisms for AAT screening purposes. Coupling with optical spectroscopy would provide additional specificity including Raman, fluorescence, chemiluminescence, circular dichroism, or other methods.

One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method for measuring cellular responses to differential stimuli using optical and/or fluidic forces, wherein the method comprises:
receiving a selection of an initial samples comprising biological cells treated with varying known levels of stimuli or analyte,
performing optical force-based measurements on the samples,
developing a response metric (RM) to describe the cellular response to the stimuli based on one or more optical or fluidic force-based parameters.

2. The method of claim 1, wherein the response metric is used to measure the response of additional unknown samples.

3. The method of claim 1, further comprising analyzing dilutions of the sample until an accurate measurement of the infectivity is determined, based upon having an RM that falls within the acceptable target value range.

4. (canceled)

5. The method of claim 1, where the optical and fluidic forces are based on laser force cytology.

6. The method of claim 1 further comprising:
comparing the response metric of an initial sample to a target value;
selecting a second sample based on the results of the first and an algorithm governing the expected or known response;
comparing the response metric of the second sample to a target value; and
selecting subsequent samples in a similar manner until a sample matching the target response metric or other defined endpoint is identified.

7. The method of claim 5, wherein the optical force-based measurements utilize laser force cytology to assess parameters comprising linear velocity, size, perimeter, size (area, diameter, volume, etc.), number of trapped cells per sample, number of beam ejected cells per sample, number of aggregates (based upon size and/or shape or other parameters), number of debris-sized particles (based upon size and/or shape or other parameters), normalized velocity, minimum x position, optical retention time, optical trapping time, optical force, optical torque, orientation, optical and fluidic dynamics, effective refractive index, eccentricity, minor axis, major axis, deformability, eccentricity deformability, minor and major axis deformability, elongation factor, compactness factor, circularity factor, images including greyscale features, whole images, image components or image derived parameters, morphology characteristics, or other laser force cytology derived parameters.

8. The method of claim 1, wherein the biological cell comprises plant cells (algal cells or others), prokaryotic cells (bacteria), eukaryotic cells, yeast, fungus, mold cells, red blood cells, neurons, egg cell (ovum), spermatozoa, white blood cells, basophils, neutrophils, eosinophils, monocytes, lymphocytes, macrophages, platelets, vesicles, exosomes, stromal cells, multicellular constructs such as spheroids, mesenchymal cells, induced pluripotent stem cells (iPSC), or cell nuclei, mitochondria, or other sub-cellular component or fraction.

9. The method claim 1, wherein the analyte comprises a virus, neutralizing serum, vaccine, oncolytic virus, protein, nucleic acid, viral vector, other virus based product, bacterium, virus that infects a bacterium, cell, or cellular product.

10. (canceled)

11. The method of claim 1, wherein the analyte is a virus and a neutralizing serum containing antibodies (viral neutralization assay), a bacterium and a neutralizing serum (bacterial neutralization assay), a toxin and antibodies in sera (toxin neutralization assay), and a virus and antiviral compound (antiviral assay), or other combination of analytes.

12.-14. (canceled)

15. The method of claim 1, wherein the cells are present in a monolayer, suspension or embedded in a matrix, wherein said matrix is comprised of alginate, gelatin, or other similar semi-solid suspension.

16. (canceled)

17. The method of claim 1, wherein the cells are sampled from an ongoing process and analyzed directly with no further incubation.

18. The method of claim 1, further comprising calibration objects.

19. The method of claim 18, wherein the calibration objects comprise beads, particles, biologics, lipids, vesicles, live cells, or fixed cells.

20. The method of claim 19, wherein said particles are spherical or non-spherical shapes sized from nanometers to millimeters composed of organic materials, polymers, metals, alloys, glass, sapphire, or diamond.

21. (canceled)

22. The method of claim 18, wherein the calibration objects are mixed with one or more samples and analyzed at the same time and wherein the calibration objects can be differentiated from cell samples based on brightfield image analysis of the cells, fluorescence measurements, or one or more optical force-based measurements.

23. (canceled)

24. A method for generating a calibration curve based on cellular response to varying concentrations of treatments and then using it to predict a sample of an unknown level:
adding treatments and incubating sample cells,
analyzing by fluidic and/or optical force-based measurements a plurality of samples having cells, and a known range of treatments to determine a response metric,
determining optimal response metric and time based on trend with dilution,
using generated data to predict future samples.

25. (canceled)

26. (canceled)

27. The method of claim 24, wherein the stimulus is viral infection and the concentration is viral titer.

28. The method of claim 24, optionally comprising additional analysis including univariate metrics, total population histogram data, subset population histogram data, K-means clustering, or PLS, PCA, neural network or other multivariate or machine learning algorithms to create a multivariate metric.

29. A method for generating a calibration curve based on cellular changes during the production of a biologic molecule or other ongoing bioprocess that correlates the cellular response to a product or cellular property of interest and then using the calibration to predict the results of a future process:
adding treatments and incubating sample cells,
analyzing by optical force-based measurements a plurality of samples having cells and a known range of product concentrations to determine a response metric;
determining optimal response metric based on trend,
using generated data to predict future samples.

30.-33. (canceled)

34. The method of claim 29, wherein the cellular property is productivity, viability, ability to produce a target molecule, differentiation state, ability to kill a specific cell type such as a tumor, ability to activate another cell type, or ability to change the biochemical state of another cell type.

35.-78. (canceled)
